(19) United States
(12) Patent Application Publication
(10) Pub. No.: US 2026/0092828 A1
(43) Pub. Date: Apr. 2, 2026

(54) Apparatus for Detecting Moisture Penetration

(71) Applicant: Mercedes-Benz Group AG, Stuttgart (DE)

(72) Inventors: Arnd BRESSEL, Dormagen (DE); Diana FRANZ, Stuttgart (DE); Frank KLEBBA, Esslingen (DE); Daniel ROTH, Herrenberg (DE)

(21) Appl. No.: 19/112,717

(22) PCT Filed: Aug. 16, 2023
(86) PCT No.: PCT/EP2023/072533

§ 371 Date: Mar. 18, 2025

(30) Foreign Application Priority Data
Sep. 19, 2022 (DE) 10 2022 003 452.2

(51) Int. Cl. G01M 3/32; G01L 19/00; G01N 33/00; H01M 10/48

(52) U.S. Cl. CPC G01M 3/3209; G01L 19/0038; G01N 33/0013; G01N 33/0016; H01M 10/48

(57) ABSTRACT

An apparatus for detecting moisture penetrating into a housing having a high-voltage component includes a gas-generating or pressure-generating element which causes gas or pressure to develop when the gas-generating or pressure-generating element contacts a liquid in droplet form. A gas or pressure sensor is arranged with the gas-generating or pressure-generating element in a common envelope element. The gas-generating or pressure-generating element is connected to the housing. The gas or pressure sensor recognizes a pressure increase or gas evolution in the common envelope element and outputs a warning signal.

### BACKGROUND AND SUMMARY OF THE INVENTION

The invention relates to an apparatus for detecting moisture penetrating into a housing having a high-voltage component according to the type defined in more detail herein.

In principle, apparatuses for detecting moisture penetration are known from the prior art. With such apparatuses, moisture penetration into a housing having a high-voltage component can be recognized. A high-voltage component may be for example components of a drive circuit of a vehicle, such as a charging device, power electronics unit or electrical or electronic engines. Furthermore, it may be housing of high-voltage batteries, which are used in electric vehicles or hybrid vehicles. With all of these components, even a little accumulation of water in droplet form can lead to critical situations, in particular caused by undesired leakage currents or electric short circuits.

An apparatus for measuring pressure and/or humidity is known from DE 10 2018 117 349 A1. Pressure and humidity can be measured via at least one sensor, comprising a capacitor having at least two electrodes. At least one dielectric layer is arranged between the electrodes. At least one electrode and/or the dielectric layer is arranged on a side facing away from a carrier material. In this case, the at least one electrode and/or the dielectric layer are arranged in a transverse direction between the carrier material and the moisture layer. Thus, a capacitance changes as a result of the liquid impinging on the dielectric layer, which can be measured via a capacitive moisture sensor.

This and further known systems can detect the liquid for example by closing an electrical contact, as long as the liquid or the medium is between the two contact surfaces. Therefore, the conductivity of the water is primarily used as a detection principle for detecting water penetration. Apparatuses of this type, for example also known as water detectors, can usually only reliably detect water penetration above a certain water quantity and in the case of sufficient conductivity.

If it is only a few drops, detecting moisture penetration is difficult or barely possible, as these drops either have to be located exactly in the electrical contact of the sensors, or have to fill a measurement space between the electrons, in the case of a measurement via conductivity. If, for example, water penetrates into the component in the form of humidity, a liquid phase is formed from condensation. This has only a very low conductivity, whereby the electrical detection is made more difficult.

A further possibility is to use pressure sensors to detect thermal events in the case of malfunctions in battery cells. Pressure sensors of such type can detect pressure fluctuations in a time-resolved manner, however they have been unsuitable to date for detecting low quantities of liquid which penetrate into an unsealed housing.

The object of the present invention is to create an apparatus for detecting the penetration of moisture which overcomes the aforementioned disadvantages.

The core of the apparatus according to the invention is formed by a gas- or pressure-generating element which causes gas or pressure to develop when it comes into contact with a liquid in droplet form, wherein a gas or pressure sensor is arranged with the gas- or pressure-generating element in a common envelope element, and the envelope element is connected to the housing via at least one access opening, wherein the gas or pressure sensor recognizes a pressure increase or gas evolution in the envelope element and outputs a warning signal. The access opening may be an interrupted support structure on the housing. Advantageously, it may be several, small-scale holes leading into the housing interior.

For example, in one embodiment, a pressure sensor, already located in the housing, for detecting a pressure increase caused by thermal events of the high-voltage component can be supplemented by a gas or pressure sensor in an envelope element, having at least one access opening into the interior of the housing. The gas- or pressure-generating element may be a chemical gas-or pressure-generating element, which reacts when coming into contact with liquid in droplet form. The envelope element itself therefore forms a type of housing, whereby the elevated pressure or gas increase is enabled in a defined region and can be detected.

The apparatus is designed for detecting moisture penetrating into a housing having a high-voltage component. The housing may be a battery housing, for example. Similarly, the apparatus can however be arranged on all components at risk from water penetration, in particular water penetration in droplet form. These can be, for example, control devices, power electronics units or electric drives.

Preferably, the pressure increase or the gas evolution can have a gradient different from that of a thermal event. Owing to the design of the gas- or pressure-generating element, an amount of gas can be generated, which leads to a drastic, i.e., very high, pressure increase inside the envelope element. This pressure increase can be detected by the gas or pressure sensor. The gas increase preferably has such a high gradient that this can be different from a gradient of a thermal event.

According to a very advantageous further development of the idea, it can be provided that the gas- or pressure-generating element has a water-soluble coating. This can prevent ageing processes of the gas- or pressure-generating element. Thus, contact of the atmosphere with reactants of the gas or pressure—generating element can be avoided. The water-soluble coating can contain or consist of readily water-soluble salts, in particular chlorides, organic substances, in particular sugar, and/or lacquer, in particular shellac. The coating can dissolve when in contact with liquid substances, in particular water, and thus enable the gas-generating reaction of the gas- or pressure-generating element.

It can be provided according to an advantageous embodiment that the gas- or pressure-generating element contains a salt compound based on carbonates and/or hydrogen carbonates. A chemical gas- or pressure-generating element of such type can cause an intensive gas evolution when it is in contact with a liquid. In particular, a mixture of the above-mentioned salt compound with the salt of a water-soluble acid, in particular an organic acid, can for example contain formates, acetates or citrates.

A further advantageous embodiment can provide that the envelope element has an equalization hole into the inner region of the housing. This can be arranged at any point on the envelope element.

It can be provided according to an advantageous embodiment that the gas- or pressure-generating element is arranged in a structural recess on the housing. As a result, a liquid droplet that occurs can accumulate in the structural recess or be channelled therein even if the total amount of liquid is very small, due to gravity and/or dynamics during operation. Thus, the occurrence of liquid in droplet form can be detected very early on.

A further advantageous embodiment can provide that the gas-or pressure-generating element and the gas or pressure sensor are arranged in two subregions of the envelope element. So, for example, the gas or pressure sensor can be spatially separated from the gas- or pressure-generating element. In this case, the gas- or pressure-generating element can be arranged at a position which is particularly susceptible to moisture or can be reached particularly early by water droplets. In contrast, the gas or pressure sensor can be arranged at another position, which is specifically protected from moisture or the formation of droplets.

A further advantageous embodiment can provide that the subregions are connected to each other via at least one supply line. Therefore, it is ensured that the elevated pressure increase, or gas increase, can be noticed equally in all subregions. The supply line can produce a gas-carrying and/or pneumatic connection between the subregions.

It can be provided according to an advantageous embodiment that the gas or pressure sensor is arranged in a region that is elevated in relation to a housing upper edge. So, the gas or pressure sensor can be protected from moisture and thus from damage.

In a further embodiment, at least one further sensor can be comprised for measuring contents of carbon dioxide and/or residual oxygen. Therefore, both thermal events can be detected inside the housing, in particular events at individual battery cells due to degradation of oxygen or formation of carbon dioxide. Furthermore, the carbon dioxide generated by the gas-or pressure-generating element or a displacement of atmospheric oxygen caused by this can be detected. Similarly, this function can be integrated into the gas or pressure sensor. In the case of a thermal event, for example detection of the event by the sensors can be ensured via the access opening or the equalization hole.

Further advantages of the apparatus according to the invention furthermore result from the remaining dependent claims and will become clear through the exemplary embodiments which are described in more detail with reference to the figures below.

### BRIEF DESCRIPTION OF THE DRAWINGS

FIG. 1 shows a possible embodiment of the apparatus;

FIG. 2 shows a further possible embodiment of the apparatus; and

FIG. 3 shows a further possible embodiment of the apparatus.

### DETAILED DESCRIPTION OF THE DRAWINGS

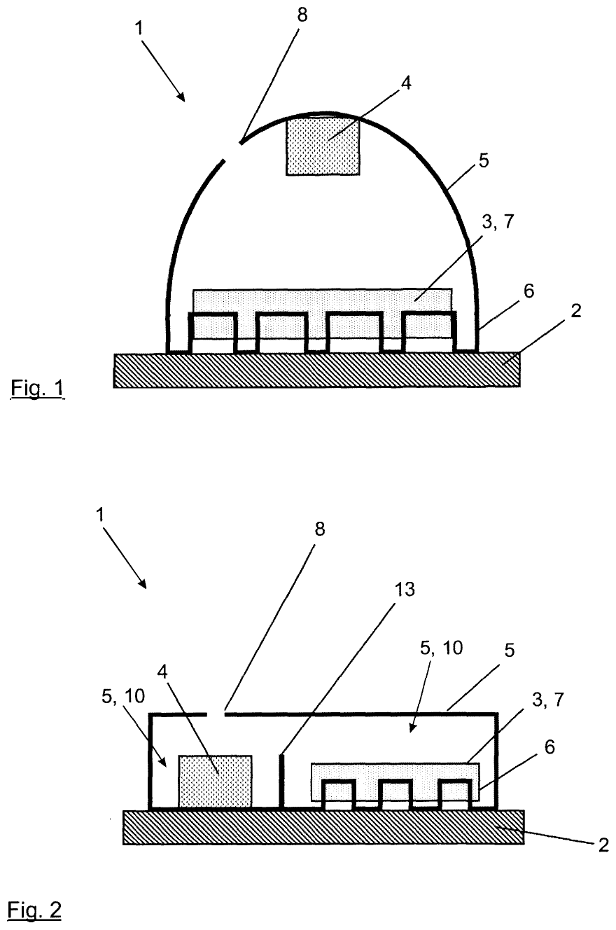

of FIG. 1, a possible embodiment of the apparatus 1 is shown. Inside an envelope element 5, which is designed in the form of a hood in this embodiment, a gas or pressure sensor 4 is arranged in the upper region and a gas-or pressure-generating element 3 is arranged in the base region. The gas-or pressure-generating element 3 is directly connected to at least one access opening 6, which creates a connection to the housing 2, in which a high-voltage component is arranged. In the upper region, an equalization hole 8 is arranged on the envelope element 5. The embodiment would similarly be conceivable without this equalization hole 8. When water enters in droplet form, the gas- or pressure-generating element 3 reacts and generates a significant pressure increase inside the envelope element 5. This can be detected by the sensor 4. When such an event is detected, a targeted message can be sent, for example to a vehicle driver or an operator.

A further embodiment variant of the apparatus 1 can be seen in FIG. 2. The same components are thus provided with the same reference numerals, so this does not have to be discussed in further detail. In contrast to FIG. 1, a combination is shown here having sensors that are already structurally attached to the housing 2 or to circuit boards. The envelope element 5 can be designed here as a type of attachment hood of a conventional sensor. A separating wall 13 is provided inside the envelope element 5, which prevents direct access of water to the gas or pressure sensor 4. Therefore, the envelope element 5 can be divided into two subregions.

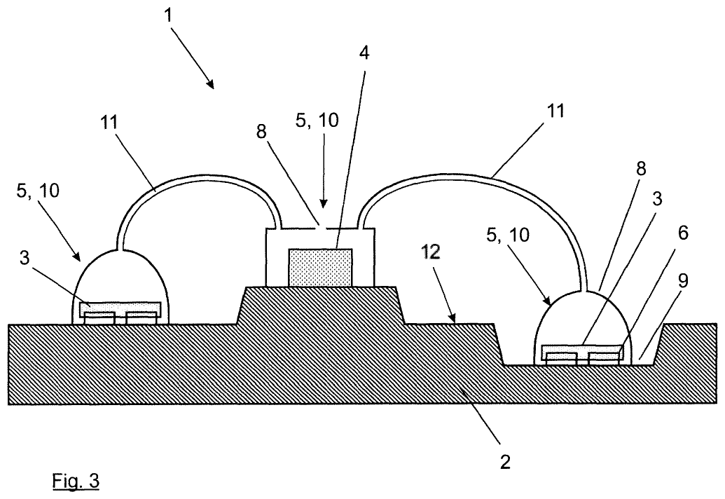

FIG. 3 shows a further embodiment of the apparatus 1, wherein the gas or pressure sensor 4 is arranged on a region that is elevated in relation to a housing upper edge 12. Thus, the sensor 4 can be protected against any liquid access, at least temporarily. In this embodiment, the gas- or pressure-generating element 3 is arranged spatially separate from the sensor 4. Consequently, the envelope element 5 has different subregions 10 which are connected to each other via supply lines 11. These supply lines 11 are gas-carrying and/or pneumatic connections and can be formed from tubes or hoses. Optionally, one of the gas or pressure sensors 4 can be arranged in a structural recess 9 on the housing 2. So, liquid droplets moved by gravity and/or dynamics of the operation can collect in this region so that a warning can be output even when the total liquid volume is low.

Clearly, the described embodiment variants of the apparatus 1 can also be combined among themselves in FIGS. 1 to 3, so that different possibilities arise. For example, the embodiment according to FIG. 3 can take place without mounting the gas- or pressure-generating element 3 in a recess. Similarly, this embodiment can only have two subregions 10, for example. Furthermore, clearly the form of the envelope element 5 is not limited to the illustrated forms.

With the provided apparatus 1, in particular high-voltage components can therefore be protected against damage caused by leakage currents and short circuits. Similarly, a user can be protected from short circuits on touchable surfaces. As a warning is issued even at very low liquid levels in droplet form, cost-intensive components, especially batteries or electric drives, can be protected or, if necessary, dried and repaired before they become unusable. The apparatus can also be designed in a cost-effective manner, when sensors that are already present in the battery are used for detection. Retrofitting to models already in series is similarly conceivable.

1.-9. (canceled)

10. An apparatus (1) for detecting moisture penetrating into a housing (2) having a high-voltage component, comprising:
a gas-generating or pressure-generating element (3) which causes gas or pressure to develop when the gas-generating or pressure-generating element (3) contacts a liquid in droplet form; and
a gas or pressure sensor (4) arranged with the gas-generating or pressure-generating element (3) in a common envelope element (5);
wherein the gas-generating or pressure-generating element (3) is connected to the housing (2) and wherein the gas or pressure sensor (4) recognizes a pressure increase or gas evolution in the common envelope element (5) and outputs a warning signal.

11. The apparatus (1) according to claim 10, wherein the pressure increase or the gas evolution has a first gradient that is different from a second gradient of a thermal event.

12. The apparatus (1) according to claim 10, wherein the gas-generating or pressure-generating element (3) has a water-soluble coating (7).

13. The apparatus (1) according to claim 10, wherein the gas-generating or pressure-generating element (3) contains a salt compound based on carbonates and/or hydrogen carbonates.

14. The apparatus (1) according to claim 10, wherein the common envelope element (5) has an equalization hole (8).

15. The apparatus (1) according to claim 10, wherein the gas-generating or pressure-generating element (3) is disposed in a structural recess (9) on the housing (2).

16. The apparatus (1) according to claim 10, wherein the gas-generating or pressure-generating element (3) and the gas or pressure sensor (4) are disposed in respective subregions (10) of the common envelope element (5).

17. The apparatus (1) according to claim 16, wherein the respective subregions (10) are connected to each other via a supply line (11).

18. The apparatus (1) according to claim 16, wherein the gas or pressure sensor (4) is disposed in a region that is elevated in relation to a housing upper edge (12).
